(19) United States
(12) Patent Application Publication
(10) Pub. No.: US 2026/0108713 A1
(43) Pub. Date: Apr. 23, 2026

(54) HEADACHE STICK

(71) Applicant: Jonathan Lawrence Rakofsky, Alexandria, VA (US)

(72) Inventor: Jonathan Lawrence Rakofsky, Alexandria, VA (US)

(21) Appl. No.: 19/085,218

(22) Filed: Mar. 20, 2025

Related U.S. Application Data
(60) Provisional application No. 63/710,934, filed on Oct. 23, 2024.

(51) Int. Cl. A61M 31/00; A61F 13/15; A61F 13/38; A61F 15/00; A61K 9/00; A61K 31/045; A61K 31/167; A61M 19/00

(52) U.S. Cl. CPC A61M 31/00; A61F 13/38; A61F 15/001; A61K 9/0043; A61K 31/045; A61K 31/167; A61M 19/00; A61F 2013/15365; A61M 2210/0618

(57) ABSTRACT

A drug delivery device for administering a drug for the treatment of pain includes an intranasal applicator including a sterile body having a first end including a polypropylene handle, and a second end including an elliptical shaped hydrophilic foam tip. The drug delivery device includes a selected amount of the drug comprising a 4% lidocaine and 0.5% menthol solution. The hydrophilic foam tip is pre-dosed with the selected amount of the drug for administration to nasal mucosa of a user.

### CROSS-REFERENCE TO RELATED APPLICATIONS

This application claims the benefit of U.S. Provisional Patent Application Ser. No. 63/710,934, filed on Oct. 23, 2024, the entire contents of which is incorporated by reference herein in its entirety.

### FIELD OF THE INVENTION

The present invention relates generally to drug delivery devices, and more particularly, to an intranasal applicator that is pre-dosed with a selected amount of a drug including a lidocaine and menthol solution for administration to the nasal mucosa for fast-acting treatment of headache pain. The present invention is also configured to be administered intranasally, as an alternative to intravenous (IV), intramuscular (IM) injection and/or oral administration such that the drug can be conveniently implemented at home, at a hospital or at a doctor's office.

### BACKGROUND OF THE INVENTION

Headaches including migraines are a common ailment that the vast majority of people suffer from at some point during their life. Typically, when a person suffers from a headache, over the counter pain relievers are self-administered orally via a capsule, tablet or a liquid. However, if headache symptoms are severe, including pain, nausea, and/or sensitivity to light, or the headache persists for an extended period of time, a person may choose to visit the emergency room (ER). At the ER, a doctor may select from a variety of drugs and forms of administration to alleviate the symptoms of the headache. For example, drugs can include nonsteroidal anti-inflammatory drugs (NSAIDs), corticosteroids, anti-nausea drugs (antiemetics), and/or a nerve block. Forms of administration can include IV, IM injection, and/or oral.

While these drugs and forms of administration are effective, the amount of time that it takes for the symptoms of a headache to be alleviated after administration can vary, and symptoms may take an extended period of time to be resolved. For example, if an oral drug is administered, it may take up to one hour to become effective. Moreover, if the patient is suffering from headache symptoms including nausea, the patient may vomit. Therefore, administration of an oral drug may cause the patient to vomit again, causing additional discomfort.

Although administration of a drug via IV or IM injection can alleviate headache symptoms faster than administration of an oral drug, IV and IM administration can be uncomfortable for certain patients due to components such as needles that are required to puncture the patient's skin. Because of this, certain patients may wish to select an alternative form of treatment. Further, patients may find it difficult or inconvenient to have to be transported to an emergency room for care.

Accordingly, there is a need for a solution to at least one of the aforementioned problems. For instance, there is an established need for preferably a drug delivery device including an intranasal applicator that is pre-dosed with a selected amount of a drug, for example, a lidocaine and menthol solution for administration to the nasal mucosa for fast-acting treatment of headache pain. There is also a need for a drug that is administered intranasally, as an alternative to IV, IM injection and/or oral administration such that the drug can be conveniently implemented at home, at a hospital or at a doctor's office.

### SUMMARY OF THE INVENTION

The present invention is directed to a drug delivery device, including an intranasal applicator that is pre-dosed with a selected amount of a drug including a lidocaine and menthol solution for administration to the nasal mucosa of a user for fast-acting treatment of headache pain. The applicator administers the drug intranasally as an alternative to IV, IM injection and/or oral administration such that the drug can be conveniently implemented at home, at a hospital or at a doctor's office.

In a first implementation of the invention, a drug delivery device for administering a drug for the treatment of pain includes an intranasal applicator including a sterile body having a first end including a polypropylene handle, and a second end including an elliptical shaped hydrophilic foam tip. The drug delivery device includes a selected amount of the drug comprising a 4% lidocaine and 0.5% menthol solution. The hydrophilic foam tip is pre-dosed with the selected amount of the drug for administration to nasal mucosa of a user.

In a second aspect, a drug delivery kit for the treatment of pain includes a drug delivery device including an intranasal applicator including a sterile body having a first end including a polypropylene handle, and a second end including an elliptical shaped hydrophilic foam tip. The kit may include a selected amount of the drug comprising lidocaine and menthol solution. The kit may include foil packaging configured for storing the drug delivery device. The hydrophilic foam tip can be pre-dosed with the selected amount of the drug for administration to nasal mucosa of a user.

In another aspect, the treatment of pain may include headache pain.

In another aspect, the handle of the intranasal applicator may include a length of 4 inches. In some embodiments, the handle may be configured to provide stability and control during application.

In another aspect, the hydrophilic foam tip can be 5 mm in size to fit into an average adult size nostril which ranges from 10-12 mm.

In another aspect, the hydrophilic foam tip can be nasal shaped.

In another aspect, the hydrophilic foam tip may include a foam swab. In some embodiments, the hydrophilic foam tip can be highly absorbent.

In another aspect, the hydrophilic foam tip can include a reservoir of the drug comprising the lidocaine and menthol solution contained within or on the foam tip.

In another aspect, the hydrophilic foam tip can be saturated with the drug.

In another aspect, the hydrophilic foam tip can be 95% to 100% saturated with the drug.

In another aspect, the foil packaging may include a pre-determined amount of the drug disposed within an interior of the foil packaging that is in contact with the hydrophilic foam tip.

In another aspect, one or a plurality of drug delivery devices can be disposed and stored within the interior of the foil packaging.

In another aspect, the foil packaging may be sterile.

In another aspect, the foil packaging can prevent the hydrophilic foam tip from drying out and assists in maintaining a moist or saturated hydrophilic foam tip.

In another aspect, the foil packaging can include a frangible or perforated section for easy tearing of the foil packaging to remove the intranasal applicator from the foil packaging.

In another aspect, a method of intranasal delivery of a drug for the treatment of headache pain can include inserting a drug delivery device including an applicator into a nasal cavity, the applicator including a sterile body having a first end including a polypropylene handle, and a second end including an elliptical shaped hydrophilic foam tip pre-dosed with a selected amount of a drug comprising a lidocaine and menthol solution; contacting nasal mucosa with the hydrophilic foam tip for a selected period of time; and removing the applicator from the nasal cavity.

In another aspect, the hydrophilic foam tip can be pre-dosed with the selected amount of the drug comprising a 4% lidocaine and 0.5% menthol solution.

In another aspect, contacting the nasal mucosa with the hydrophilic foam tip for a selected period of time can include about 1-2 minutes.

In another aspect, the applicator can be inserted into the nasal cavity of a respective side where the headache pain is located.

In another aspect, the applicator can be inserted into left and/or right nasal cavities. In some embodiments, the applicator can be inserted into the left and right nasal cavities if the headache pain is located on both sides of a user's head.

In some embodiments, the hydrophilic foam tip can be pre-dosed with the selected amount of the drug to enhance patient comfort and compliance while minimizing risk of systemic side effects.

In some embodiments, the drug delivery device can be configured for precise and controlled administration of the drug to the nasal mucosa to depolarize sphenopalatine ganglion to enhance a patient's comfort and compliance during medical procedures. In some embodiments, the drug delivery device can be configured for safe and effective pain relief.

In some embodiments, the drug delivery device can be configured for application in pain management during medical procedures such as nasal surgeries or diagnostic tests, treatment of acute and chronic nasal pain and headache conditions, and/or emergency medical care for rapid pain relief.

In some embodiments, the drug delivery device can be configured to provide a user-friendly, precise, and effective method for delivering lidocaine to the nasal mucosa. In some embodiments, the design of the drug delivery device and the method of use may provide healthcare providers and patients with a reliable tool for managing nasal pain and discomfort.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents an isometric view of a drug delivery device for use in administering a drug to treat headaches in accordance with a first illustrative embodiment of the present invention;

FIG. 2 presents an isometric view of the drug delivery device as illustrated in FIG. 1 that is disposed adjacent to a nose of a user prior to administration of the drug;

FIG. 3 presents an isometric view of the drug delivery device as illustrated in FIG. 1 disposed within a nasal passage of the user to administer the drug;

FIG. 4 presents an enlarged isometric view of an end of drug delivery device as illustrated in FIG. 1; and

FIG. 5 presents an isometric view of the drug delivery device as illustrated in FIG. 1 that is stored within a foil packaging containing a plurality of drug delivery devices to form a kit.

Like reference numerals refer to like parts throughout the several views of the drawings.

### DETAILED DESCRIPTION

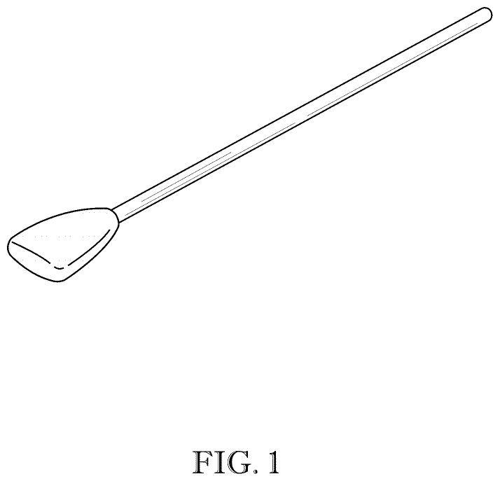

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, the present invention is directed toward a drug delivery device (e.g., headache stick) including an intranasal applicator that is pre-dosed with a selected amount of a drug including a lidocaine and menthol solution for administration to the nasal mucosa of a user for fast-acting treatment of pain, including headache pain. The applicator administers the drug intranasally as an alternative to IV, IM injection and/or oral administration such that the drug can be implemented at home, at a hospital or at a doctor's office.

Referring initially to FIGS. 1-5, a drug delivery device 100, is illustrated in accordance with a first exemplary embodiment of the present invention. As shown for instance in FIG. 1, drug delivery device 100 includes an intranasal applicator 102. Applicator 102 includes a sterile body 104 that extends between an end 106 and an end 108. Body 104 includes a cylindrical cross section configuration. In some embodiments, body 104 may have alternate cross section configurations, including, but not limited to, rectangular, oval, oblong triangular, square, polygonal, irregular, uniform, non-uniform, offset, staggered, and/or tapered.

End 106 includes a handle 110 configured to provide stability and control during use/application. Handle 110 is manufactured from a thermoplastic polymer, for example, polypropylene. In some embodiments, handle 110 is alternatively manufactured from paper, bamboo, plastic, rubber and/or wood.

Handle 110 includes a selected length of 4 inches. In some embodiments, handle 110 includes a selected length in a range of 2 to 8 inches or 4 to 6 inches. In some embodiments, handle 110 includes a selected thickness. In some embodiments, handle 110 includes a selected thickness in a range of 3 millimeters (mm) to 8 mm or 4 mm to 6 mm.

In some embodiments, one or more exterior surfaces of handle 110 may have various surface configurations to enhance engagement with a user's fingers and/or hand during use including, but not limited to smooth, rough, dimpled, threaded and/or textured.

In some embodiments, one or more exterior surfaces of handle 110 may include indicia, for example, units of measurement to assist in visually indicating a distance that applicator 102 has travelled through a nasal cavity of a user. In some embodiments, handle 110 is manufactured in a selected color. In some embodiments, handle 110 is manufactured in a selected color including white, tan, pink, red, orange, yellow, blue, green, purple, gray, brown and/or black.

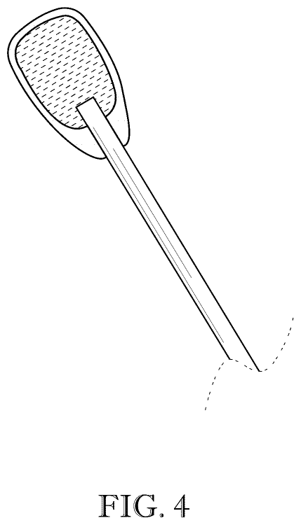

As shown in FIG. 4, end 108 includes a tip 112. Tip 112 is configured to be pre-dosed with a selected amount of a drug 114 for administration to the nasal mucosa of a user for fast-acting treatment of pain, including headache pain. It is to be understood that tip 112 includes a swab. In some embodiments, tip 112 is pre-dosed with a selected amount of drug 114 to enhance patient comfort and compliance while minimizing risk of systemic side effects.

Tip 112 includes an elliptical shape and is manufactured from a highly absorbent hydrophilic foam. In some embodiments, tip 112 is nasal shaped. In some embodiments, tip 112 includes an alternative shape including oval, circular, square, rectangular, conical, triangular, pentagonal, hexagonal, or octagonal. In some embodiments, tip 112 is alternatively manufactured from cotton, rayon, polyester and/or flocked material.

Tip 112 includes a reservoir 116 of drug 114 such that drug 114 is retained within or on tip 112. In some embodiments, tip 112 is saturated with the pre-dosed amount of drug 114. In some embodiments, tip 112 is 95% to 100% saturated with the pre-dosed amount of drug 114. In some embodiments, tip 112 is 95%, 96%, 97%, 98%, 99% or 100% saturated with the pre-dosed amount of drug 114.

Tip 112 is manufactured in a selected size of 5 mm to fit into an average adult size nostril which can range from about 10-12 mm. In some embodiments, tip 112 is alternatively manufactured in a selected size in a range of 3 to 8 mm or 4 to 6 mm.

As described herein, tip 112 is pre-dosed with a selected amount of drug 114. Drug 114 includes one or more selected active ingredients including lidocaine and menthol. In some embodiments, drug 114 includes lidocaine hydrochloride as the active ingredient. The active ingredient lidocaine is a widely used local anesthetic known for its efficacy in pain relief. Intranasal administration offers advantages such as rapid onset of action and avoidance of first-pass metabolism. When lidocaine is administered into the nose, it is quickly absorbed into the bloodstream and brain, where it may temporarily numb the sphenopalatine ganglion (SPG) cells. The SPG is a group of nerve cells located behind the nose that are associated with headache pain and the trigeminal nerve, which is also involved in headaches. Lidocaine also controls migraine pain by inactivating or desensitizing the intracranial nociceptors that contribute to the vasodilation of the cerebral vasculature, producing migraine. Lidocaine administered on nasal or oral mucosa produces prompt analgesia without serious adverse reactions in patients. The active ingredient menthol (e.g., peppermint oil), has been found to block the protein called TRPM8 which has been correlated with migraine-like symptoms.

In some embodiments, drug 114 includes a 4% lidocaine and 0.5% menthol solution. In some embodiments, drug 114 includes a range of 1% to 10% lidocaine, and/or a range of 0.2% to 0.9% menthol solution. In some embodiments, drug 114 includes 1%, 2%, 3%, 4%, 5%, 6%, 7%, 8%, 9% or 10% lidocaine, and/or 0.2%, 0.3%, 0.4%, 0.5%, 0.6%, 0.7%, 0.8%, or 0.9% menthol.

In some embodiments, drug 114 alternatively or additionally includes articaine, bupivacaine, cinchocaine/dibucaine, etidocaine, levobupivacaine, lidocaine/lignocaine, mepivacaine, prilocaine, ropivacaine, and/or trimecaine in a range of 1% to 10%.

In some embodiments, drug 114 alternatively or additionally includes one or more triptans including sumatriptan, naratriptan, zolmitriptan, rizatriptan, almotriptan, frovatriptan, and/or eletriptan in a range of 1% to 10%.

In some embodiments, drug 114 alternatively or additionally includes nonsteroidal anti-inflammatory drugs (NSAIDs), corticosteroids, anti-nausea drugs (antiemetics), and/or a nerve block.

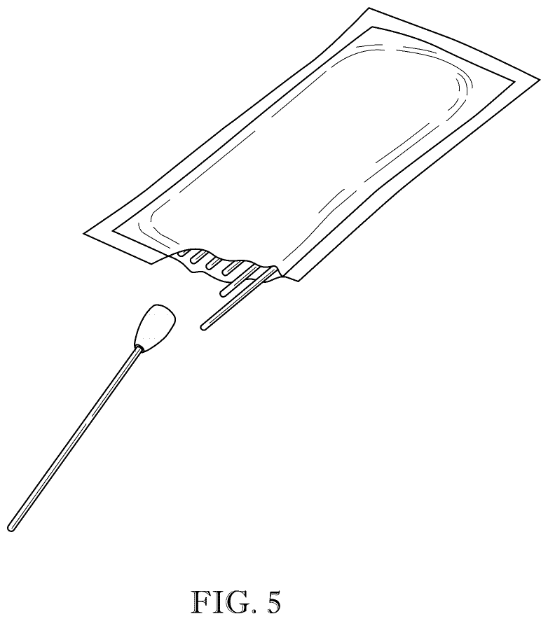

As shown in FIG. 5, drug delivery device 100 can be stored within an interior 120 of a sterile foil packaging 118 to form a kit 122. Packaging 118 is configured to store one or a plurality of drug delivery devices 100. In some embodiments, packaging 118 includes a pre-determined amount of drug 114 disposed within interior 120 such that drug 114 is in contact with a plurality of tips 112. In some embodiments, packaging 118 prevents the plurality of tips 112 from drying out and assists in maintaining the plurality of tips 112 moisture or saturation. In some embodiments, packaging 118 can include one or more frangible or perforated sections for easy tearing of packaging 118 to remove drug delivery device(s) 100 from packaging 118.

In typical application, drug delivery device 100 is implemented for pain management at home or during medical procedures such as nasal surgeries or diagnostic tests, treatment of acute and chronic nasal pain and headache conditions, and/or emergency medical care for rapid pain relief. It is to be understood that the term headache or headache conditions can include a migraine.

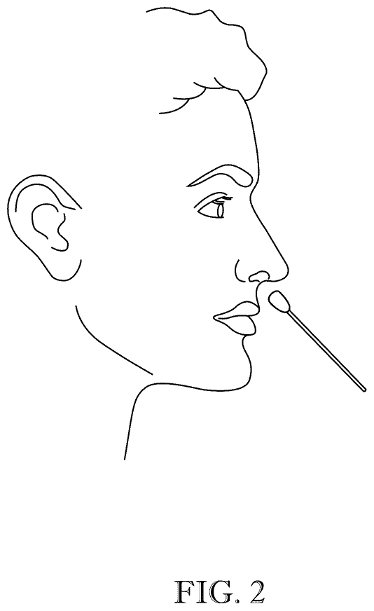

As illustrated in FIGS. 2-3 and 5, a method of intranasal delivery of a drug for the treatment of headache pain is provided. In some embodiments, the user/patient U/P is suffering from a frontal or temporal headache or migraine. The method includes the step of inserting drug delivery device 100 including applicator 102 that is pre-dosed with a selected amount of drug 114 comprising a lidocaine and menthol solution into a selected nasal cavity NC of a nose N of a U/P. In some embodiments, tip 112 is pre-dosed with the selected amount of drug 114 comprising a 4% lidocaine and 0.5% menthol solution. In some embodiments, applicator 102 is inserted into the NC of a respective side of the N where the headache pain is located. In some embodiments, applicator 102 is inserted into the left NC or the right NC. In some embodiments, applicator 102 is inserted into the left NC and the right NC if the headache pain is located on both sides of a U's/P's head.

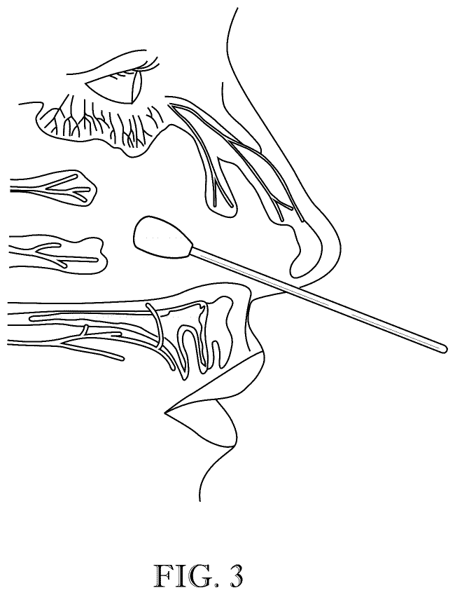

The method includes the step of contacting nasal mucosa NM (FIG. 3) with tip 112 for a selected period of time. In some embodiments, the selected period of time is about 1 to 6 minutes. In some embodiments, the selected period of time is about 1 to 2 minutes. The method includes the step of removing applicator 102 from the NC.

In some embodiments, prior to the step of inserting drug delivery device 100 into a selected NC of the N of the U/P, drug delivery device 100 is stored in foil packaging 118. Foil packaging 118 is opened and one or more drug delivery devices 100 are removed from foil packaging 118, as shown in FIG. 5.

In some embodiments, administration of drug 114 to the NM becomes effective to treat headache pain in a period of time of less than 10 minutes. In some embodiments, the period of time for drug 114 to become effective after administration to the NM is in a range of 1 to 10 minutes, 2 to 8 minutes or 4 to 6 minutes. In some embodiments, the period of time for drug 114 to become effective after administration to the NM is about 1, 2, 3, 4, 5, 6, 7, 8, 9 or 10 minutes.

In some embodiments, drug delivery device 100 is configured for precise and controlled administration of drug 114 to the NM to depolarize the SPG to enhance a U's/P's comfort and compliance during medical procedures. In some embodiments, drug delivery device 100 is configured for safe and effective pain relief.

In some embodiments, drug delivery device 100 is configured to provide a user-friendly, precise, and effective method for delivering lidocaine to the NM. In some embodiments, the design of drug delivery device 100 and method provides healthcare providers and patients with a reliable tool for managing nasal pain and discomfort.

In some embodiments, at home use of drug delivery device 100 can reduce the number of patients in the ER and may help to decongest the ERs across the United States. In some embodiments, at home use of drug delivery device 100 could potentially avoid millions of dollars wasted on ER visits.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A drug delivery device for administering a drug for the treatment of pain, the drug delivery device comprising:
an intranasal applicator including a sterile body having a first end including a polypropylene handle, and a second end including an elliptical shaped hydrophilic foam tip; and
a selected amount of the drug comprising a 4% lidocaine and 0.5% menthol solution,
wherein the hydrophilic foam tip is pre-dosed with the selected amount of the drug for administration to nasal mucosa of a user.

2. The drug delivery device of claim 1, wherein the treatment of pain comprises the treatment of headache pain.

3. The drug delivery device of claim 1, wherein the handle of the intranasal applicator has a length of 4 inches.

4. The drug delivery device of claim 1, wherein the hydrophilic foam tip is 5 mm.

5. The drug delivery device of claim 1, wherein the hydrophilic foam tip is nasal shaped.

6. The drug delivery device of claim 1, wherein the hydrophilic foam tip includes a foam swab.

7. The drug delivery device of claim 1, wherein the hydrophilic foam tip includes a reservoir of the drug comprising the lidocaine and menthol solution contained within or on the foam tip.

8. The drug delivery device of claim 1, wherein the hydrophilic foam tip is saturated with the drug.

9. The drug delivery device of claim 1, wherein the hydrophilic foam tip is 95% to 100% saturated with the drug.

10. A drug delivery kit for the treatment of pain comprising:
the drug delivery device of claim 1; and,
foil packaging configured for storing the drug delivery device.

11. The drug delivery kit of claim 10, wherein the foil packaging includes a pre-determined amount of the drug disposed within an interior of the foil packaging that is in contact with the hydrophilic foam tip.

12. The drug delivery kit of claim 10 wherein a plurality of drug delivery devices are disposed and stored within the interior of the foil packaging.

13. The drug delivery kit of claim 10, wherein the foil packaging is sterile.

14. The drug delivery kit of claim 10, wherein the foil packaging is configured to prevent the hydrophilic foam tip from drying out and to assist in maintaining a moist or saturated hydrophilic foam tip.

15. The drug delivery kit of claim 10, wherein the foil packaging includes a frangible or perforated section for easy tearing of the foil packaging to remove the intranasal applicator from the foil packaging.

16. A method of intranasal delivery of a drug for the treatment of headache pain comprising inserting a drug delivery device including an applicator into a nasal cavity, the applicator including a sterile body having a first end including a polypropylene handle, and a second end including an elliptical shaped hydrophilic foam tip pre-dosed with a selected amount of a drug comprising a lidocaine and menthol solution;
contacting nasal mucosa with the hydrophilic foam tip for a selected period of time; and
removing the applicator from the nasal cavity.

17. The method of claim 16, wherein the hydrophilic foam tip is pre-dosed with the selected amount of the drug comprising a 4% lidocaine and 0.5% menthol solution.

18. The method of claim 16 wherein the contacting of the nasal mucosa with the hydrophilic foam tip for a selected period of time is from about 1 to about 2 minutes.

19. The method of claim 16, wherein the applicator is inserted into the nasal cavity of a respective side where the headache pain is located.

20. The method of claim 16, wherein the drug delivery device is configured for precise and controlled administration of the drug to the nasal mucosa to depolarize sphenopalatine ganglion.
